(19) United States
(12) Patent Application Publication
(10) Pub. No.: US 2009/0068153 A1
(43) Pub. Date: Mar. 12, 2009

(54) CELL COMPOSITION FOR TISSUE REGENERATION

(76) Inventors: Francesca P. Vitelli, Houston, TX (US); David A. Wolf, Houston, TX (US); Donnie Rudd, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/850,775

(22) Filed: Sep. 6, 2007

(51) Int. Cl. C12N 5/08; A61K 35/50

(52) U.S. Cl. 424/93.7; 435/366

(57) ABSTRACT

A method of extracting human progenitor cells from perivascular tissue of human umbilical cord. The extracted cells are then co-cultured with hematopoetic stem cells and are useful to grow and repair human tissues including bone. Also included are related methods and compositions related thereto.

### FIELD OF INVENTION

This invention focuses on the harvesting of a population of rapidly proliferating human cells from the connective tissue of the umbilical cord, methods related to co-culturing these cells with hematopoetic stem cells, compositions related thereto, and useful for various cell-based therapies.

### BACKGROUND OF THE INVENTION

The obtaining of therapeutic cell mixtures from Wharton's Jelly is well known. However, in each instance it has been considered critical to insure that any trace of cord blood was eliminated, an expensive and time-consuming procedure. The present invention is not burdened with this problem. The present invention co-cultures the cells derived from Wharton's Jelly with hematopoetic stem cells.

The umbilical cord is one of the first structures to form following gastrulation (formation of the three embryonic germ layers). As folding is initiated, the embryonic disc becomes connected, by the primitive midgut (embryonic origin) to the primitive yolk sac (extra-embryonic origin) via the vitelline and allantoic vessels which in turn develop to form the umbilical vessels (Haynesworth et al., 1998; Pereda and Motta, 2002; Tuchmann-Duplessis et al., 1972). These vessels are supported in, and surrounded by, what is generally considered a primitive mesenchymal tissue of primarily extra-embryonic derivation called Wharton's Jelly (WJ) (Weiss, 1983). From this early stage, the umbilical cord grows, during gestation, to become the 30-50 cm cord seen at birth. It can be expected therefore, that WJ contains not only the fibroblast-like, or myo-fibroblast-like cells which have been described in the literature (see below), but also populations of progenitor cells which can give rise to the cells of the expanding volume of WJ necessary to support the growth of the cord during embryonic and fetal development.

WJ was first described by Thomas Wharton, who published his treatise Adenographia in 1656. (Wharton T W. Adenographia. Translated by Freer S. (1996). Oxford, U.K.: Oxford University Press, 1656; 242-248). It has subsequently been defined as a gelatinous, loose mucous connective tissue composed of cells dispersed in an amorphous ground substance composed of proteoglycans, including hyaluronic acid (Schoenberg et al., 1960), and different types of collagens (Nanaev et al., 1997). The cells dispersed in the matrix have been described as “fibroblast-like” that are stellate in shape in collapsed cord and elongate in distended cord (Parry, 1970). Smooth muscle cells were initially observed within the matrix (Chacko and Reynolds, 1954), although this was disputed by Parry (1970) who described them as somewhat “unusual fibroblasts” which superficially resemble smooth muscle cells. Thereafter, little work had been done on characterizing these cells until 1993 when Takechi et al. (1993) performed immunohistochemical investigations on these cells. They described the cells as “fibroblast-like” that were “fusiform or stellate in shape with long cytoplasmic processes and a wavy network of collagen fibres in an amorphous ground substance” (Takechi et al., 1993). For the immunohistochemical staining, they used primary antibodies against actin and myosin (cytoplasmic contractile proteins), vimentin (characteristic of fibroblasts of embryonic mesenchyme origin) and desmin (specific to cells of myogenic origin) in order to determine which types of myosin are associated with the WJ fibroblasts. They observed high levels of chemically extractable actomyosin; and although fibroblasts contain cytoplasmic actomyosin, they do not stain for actin or myosin, whereas the WJ fibroblasts stained positively for both. Additionally, positive stains for both vimentin and desmin were observed leading to the conclusion that these modified fibroblasts in WJ were derived from primitive mesenchymal tissue (Takechi et al., 1993). A subsequent, more recent study by Nanaev et al. (1997) demonstrated five steps of differentiation of proliferating mesenchymal progenitor cells in pre-term cords. Their findings supported the suggestion that myofibroblasts exist within the WJ matrix. The immunohistochemical characterization of the cells of WJ, shows remarkable similarities to that of pericytes which are known to be a major source of osteogenic cells in bone morphogenesis and can also form bone nodules referred to as colony forming unit-osteoblasts (CFU-O) (Aubin, 1998) in culture (Canfield et al., 2000).

Recent publications have reported methods to harvest cells from UC, rather than UC blood. Mitchell et al. (Mitchell et al., 2003) describe a method in which they first remove and discard the umbilical vessels to harvest the remaining tissue. The latter, which will include both the remaining WJ (some of which will have been discarded with the vessels, since the umbilical vessels are entirely enveloped in WJ) and the amniotic epithelium, is then diced to produce small tissue fragments that are transferred to tissue culture plates. These tissue fragments are then used as primary explants from which cells migrate onto the culture substratum.

In another publication, Romanov et al. (2003) indicate they were successful in isolating mesenchymal stem cell-like cells from cord vasculature, although they also indicate their cultures do not contain cells from WJ. Specifically, they employ a single, 15 min, collagenase digestion from within the umbilical vein, which yields a mixed population of vascular endothelial and sub-endothelial cells. Romanov et al. show that sparse numbers of fibroblast-like cells appear from this cell harvest after 7 days.

It is an object of the present invention to provide a cell population comprising human progenitor cells co-cultured with hematopoetic stem cells. It is a further object of the present invention to provide human cell mixture that can be useful therapeutically.

### SUMMARY OF THE INVENTION

There has now been devised a procedure for extracting cells from Wharton's jelly of human umbilical cord, which yields a unique cell population characterized by rapid proliferation, the presence of osteoprogenitor and other human progenitor cells, including immuno-incompetent cells which display neither of the major histocompatibility markers (human leukocyte antigen (HLA) double negative). The cell population when co-cultured with hematopoetic stem cells is a useful source of cells from which to grow bone and other connective tissues including cartilage, fat and muscle, and for autogenic and allogeneic transfer of progenitor cells to patients, for therapeutic purposes.

More particularly, and according to one aspect of the present invention, there is provided a Wharton's jelly extract, wherein the extract comprises human progenitor cells and is obtained by enzymatic digestion of the Wharton's jelly proximal to the vasculature of human umbilical cord, in a region usefully termed the perivascular zone of Wharton's jelly. The tissue within this perivascular zone, and from which the present progenitor cells are extracted, can also be referred to as perivascular tissue. The extraction procedure results in an extract that is essentially free from cells of umbilical cord blood, epithelial cells or endothelial cells of the UC and cells derived from the vascular structure of the cord, where vascular structure is defined as the tunicae intima, media and adventia of arteriolar or venous vessels. The resultant extract is also distinct from other Wharton's jelly extracts isolated from the bulk Wharton's jelly tissue that has been separated from the vascular structures. These cells are then co-cultured with hematopoetic stem cells to create a tissue regenerating mixture.

In a related aspect, the present invention provides a cell population obtained by culturing of the cells present in the Wharton's jelly extract and then co-culturing them with hematopoetic stem cells. The co-culturing can be accomplished in a two-dimensional system such as t-flasks or preferably a three dimensional system such as a rotating wall bioreactor.

In one embodiment, the extracted progenitor cell population is characterized as an adherent cell population obtained following culturing of the extracted cells under adherent conditions. In another embodiment, the extracted progenitor cell population is characterized as a non-adherent (or “post-adherent”) (PA) cell population present within the supernatant fraction of extracted cells grown under adherent conditions. This PA fraction is derived by transferring the supernatant of the initially plated HUCPV cells into a new T-75 flask to allow the as yet non-adhered cells to attach to the culture surface. This process is repeated with this new T-75 flask, transferring its media into another new T-75 flaks in order to harvest any remaining PA cells. This PA cell population comprises, according to another aspect of the invention, a subpopulation of progenitor cells that, when cultured under adherent conditions and then co-cultured with hematopoetic stem cells, proliferates rapidly and forms bone nodules and fat cells spontaneously. This embodiment provides a means to increase the yield of adherent cells isolated from the enzymatic digest cell population.

In another of its aspects, the present invention provides a method for producing connective tissue, including bone tissue, cartilage tissue, adipose tissue and muscle tissue, which comprises the step of subjecting the co-cultured mixture to conditions conducive to differentiation of those cells into the desired connective tissue phenotype. In this respect, the invention further provides for the use of such cells in cell-based therapies including cell transplantation-mediated treatment of medical conditions, diseases and disorders.

More particularly and according to another aspect of the invention, there is provided a composition and the use thereof in tissue engineering, comprising a cell mixture in accordance with the invention or their differentiated progeny, and a carrier suitable for delivering such cells to the chosen tissue site.

These and other aspects of the invention will now be described in greater detail with reference being had to the accompanying drawings, in which:

### DESCRIPTION OF THE FIGURES

FIG. 1 is a light micrograph representing the three distinct zones of tissue represented in the human UC;

FIG. 2 is a representative illustration of the looped vessel in the collagenase solution;

FIG. 3 is a light micrograph of the cells isolated from the WJ that have attached to the polystyrene tissue culture surface;

FIG. 4 is a light micrograph illustrating the initial formation of a CFU-O;

FIG. 5 is a light micrograph illustrating a mature CFU-O;

FIG. 6 demonstrates tetracycline-labeled CFU-O's under UV fluorescence on a 35 mm polystyrene tissue culture dish;

FIG. 7 illustrates side by side a phase-contrast light micrograph and a fluorescence micrograph of the same tetracycline-labeled CFU-o;

FIG. 8 is a scanning electron micrograph of a mature CFU-O on the tissue culture polystyrene surface;

FIG. 9 is a scanning electron micrograph of a cross-section of a CFU-O exposing the underlying matrix;

FIG. 10 is a scanning electron micrograph of the lightly mineralized collagen fibres located on the advancing edge of the CFU-O;

FIG. 11 is a scanning electron micrograph of the non-collagenous matrix (seen as globules) laid down on the polystyrene interface by differentiating osteogenic cells;

FIG. 12 is a scanning electron micrograph of heavily mineralized collagen that comprises the centre of a mature CFU-O;

FIG. 13 illustrates the flow cytometry data demonstrating that WJ-derived cells are 77.4% MHC I and MHC II negative;

FIG. 14 is a black and white reproduction of a Masson's trichome-stained transverse section of bone nodule showing the distribution of collagen within which cells have become entrapped (osteocytes), and multilayering of peripheral cells some of which are becoming surrounded by the elaborated extracellular matrix:

FIG. 15 shows the potential expansion of the adherent perivascular WJ population in relation to the expansion of the committed osteoprogenitor subpopulation and total osteoprogenitor subpopulation;

FIG. 16 shows proliferation of the perivascular WJ cells from 0-144 hours illustrating a normal growth curve with a lag phase from 0-24 hrs, log phase from 24-72 hours, and plateau phase from 72-120 hours. The doubling time during the entire culture period is 24 hours, while during log phase it is 16 hours;

FIG. 17 shows major histocompatibility complex (MHC) expression of the WJ cells shown over 5 passages, the change in their expression due to free-thawing, and subsequent expression due to reculture;

FIG. 18 shows the CFU-F frequency of HUCPV cells;

FIG. 19 shows the doubling time of HUCPV cells from P0 through P9. HUCPV cells demonstrate a relatively stable and rapid doubling time of 20 hours from P2 to P8; and

FIG. 20 shows the proliferation of HUCPV cells demonstrating that>10¹⁴ cells can be derived within 30 days of culture. With this rapid expansion, 1,000 therapeutic doses (TDs) can be generated within 24 days of culture; and

FIG. 21 shows the effects of collagenase concentration and digestion time on cell harvest.

FIG. 22 shows a perspective view of the general organization of the bioreactor of the present invention.

FIG. 23 shows a view in partial cross section through a horizontally rotated cell culture vessel illustrating an application of the present invention.

FIG. 24 is a view in cross section taken along line 23-23 of FIG. 23; and

FIG. 25 is a view in cross section taken along line 24-24 of FIG. 23.

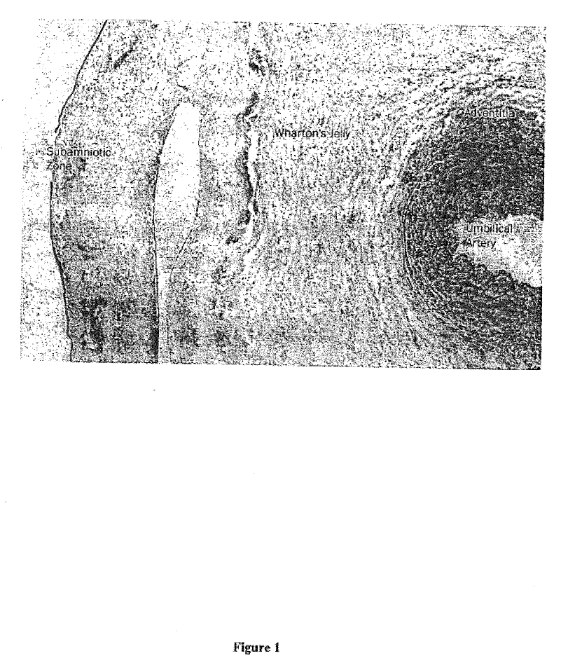

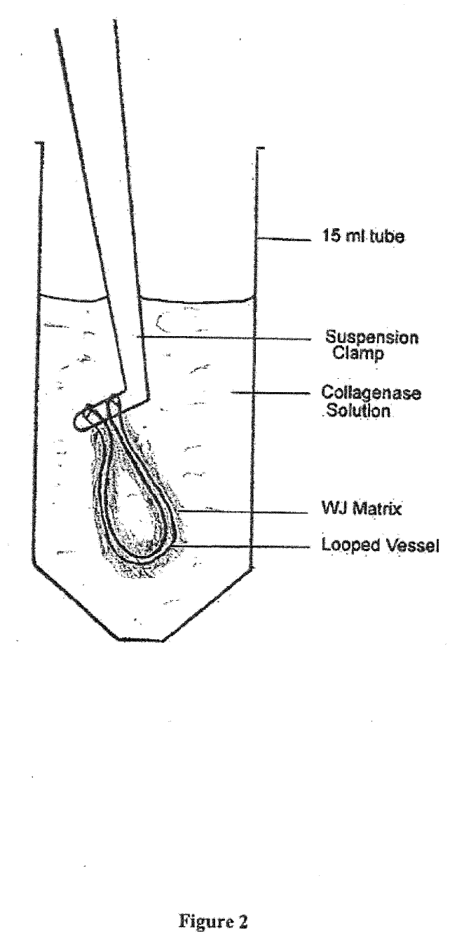

### DETAILED DESCRIPTION OF THE INVENTION

The present invention provides an extract of Wharton's jelly (WJ), as a source of a rapidly proliferating cell population comprising human progenitor cells.

For purposes of this description, the extracted cell population portion can be referred to as human umbilical cord perivascular (HUCPV) cells. The HUCPV cell population constitutes a rich source of multipotent progenitor cells that are unique in their phenotype, particularly as revealed by the variety of cell subpopulations contained therein. Also for purposes of this description, the perivascular zone of the Wharton's jelly from which the present cells are extracted can be referred to as perivascular tissue.

As used herein, the term “progenitor cells” refers to cells that will differentiate under controlled and/or defined conditions into cells of a given phenotype. “Progenitor cells” are also characterized by the ability to self-renew in addition to differentiate. This characteristic of self-renewal is referred to “proliferation”. Thus, an osteoprogenitor cell is a progenitor cell that will commit to the osteoblast lineage, and ultimately form bone tissue when cultured under conditions established for such commitment and differentiation. A progenitor cell that is “immuno-incompetent” or “non-immunogenic” is a cell having a phenotype that is negative for surface antigens associated with class I and class II major histocompatibility complexes (MHC). Such a progenitor cell is also referred to herein as an HLA double negative.

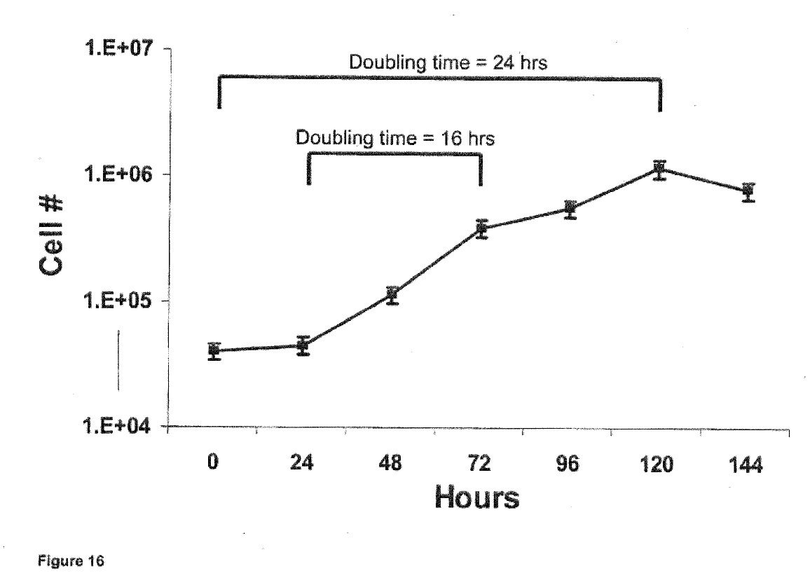

The HUCPV cell population extracted from WJ is also characterized by “rapid proliferation”, which refers to the rate at which the extracted cells will grow relative to other known progenitor cell populations, under conditions that are standard for progenitor cell expansion. As will be appreciated from the experimental results presented herein, and as shown in FIG. 16, the present progenitor cell population can double within at least about 25 hours and as quickly as 7-15 hours, and thus expands far more rapidly than other known osteoprogenitor cell populations and other progenitor cell populations extracted from WJ.

The cells and cell populations of the present invention can be obtained by extraction from WJ of human umbilical cord and then co-cultured with hematopoetic stem cells derived from cord blood or peripheral blood. Unlike the prior art, and in accordance with the present invention, the first group of such cells are extracted from the WJ that is associated with, i.e., proximal to, the exterior wall of the umbilical vasculature. The Wharton's jelly that is associated with or very near to the external surface of the cord vasculature lies within a region termed the perivascular zone, and typically remains associated with the vasculature when the vessels are excised from the cord, as is done for instance either to extract Wharton's jelly from the cord, or to extract the vessels from the cord and associated Wharton's jelly. It has remarkably been found that the Wharton's jelly within this perivascular zone, and which has typically been discarded in prior art practice, is a rich source of progenitor cells having the characteristics herein described. Accordingly, the present invention exploits the tissue from this perivascular zone of the Wharton's jelly as a source for useful human progenitor cells, termed HUCPV cells.

In the embodiments, the HUCPV cell population is characterized by the presence of progenitor cells having many markers indicative of a functional mesenchymal (non-hematopoietic) phenotype, preferably the following markers are present on these progenitor cells including CD45−, CD34−, SH2+, SH3+, Thy−1+ and CD44+. Other preferred markers may be used to identify a functional mesenchymal phenotype as well. Preferably, the population is characterized as harboring cells that are positive for 3G5 antibody, which is a marker indicative of pericytes. The extracted cell population generally is a morphologically homogeneous fibroblastic cell population, which preferably expresses alpha-actin, desmin, and vimentin, and provides a very useful source from which desired cell subpopulations can be obtained through manipulation of culturing conditions and selection based for instance on cell sorting principles and techniques.

To extract such perivascular cells from human umbilical cord, in a preferred embodiment, care is taken during the extraction process to avoid extracting cells of the umbilical cord blood, epithelial cells or endothelial cells of the umbilical cord, and cells derived from the vascular structure of the cord, where vascular structure is defined as the tunicae intima, media and adventia of arterial or venous vessels. A preferred method of obtaining an extract that is essentially free of these unwanted cells can be achieved by careful flushing and washing of the umbilical cord prior to dissection, followed by careful dissection of the vessels from within the cord. Another preferred method is by carefully pulling the vessels away from the surrounding cord tissue in which case the perivascular tissue is excised with the vessels. It will be appreciated that, with care being taken to avoid extracting these unwanted cells, they may still be present to a small extent in the resulting extract. This is acceptable provided they occur at a frequency too low to interfere with the observed results presented herein, i.e., observation of cell colonies derived from mesenchymal and specifically mesodermal origin, frequency and rapidity of formation of CFU-F, CFU-O and CFU-A, and characterization of HLA phenotypes observed in the cultured population. It is only after the HUCPV cell population is prepared that it is co-cultured with the hematopoetic cell population.

The tissue that lies within the perivascular zone is the Wharton's jelly proximal to the external wall of the umbilical vasculature, and lies typically within a zone extending to about 3 mm from the external wall of the vessels. Preferably, the target extraction zone can lie within about 2 mm, more preferably, about 1 mm from the external wall of any one of the three vessels. The extraction of WJ from this region can be readily achieved, preferably using the technique described in the examples. In the preferred embodiments disclosed in the examples the vessels are used as a carrier for the WJ, and the vessels per se are used as the substrate from which the progenitor cells are extracted. Thus, in embodiments of the invention, cord vessels bearing a thin coating of perivascular tissue are excised either preferably surgically or more preferably manually from fresh umbilical cord that has been washed thoroughly to remove essentially all cord blood contaminants. The vessels bearing the proximal perivascular tissue, or sections thereof, are then incubated at about 37° C. in an extraction medium, preferably such as phosphate buffered saline (PBS), containing an enzyme suitable for digesting the collagen matrix of the perivascular tissue in which the desired cells reside. For this purpose, digestion with a collagenase is suitable, at a preferred concentration preferably within the range from about 0.1 mg/mL to about 10.0 mg/mL or more, more preferably 0.5 mg/mL. The enzyme type, concentration and incubation time can vary, and alternative extraction conditions can be determined readily simply by monitoring yield of cell phenotype and population under the chosen conditions. For instance, in a preferred embodiment, a higher collagenase concentration of 4 mg/mL (e.g., 1-4 mg/mL) is also suitable over a shorter digestion period of about 3 hours (e.g., 1-5 hours). During the extraction, the ends of the vessels are bound, preferably tied, or clipped, off and can be suspended above the extraction medium to avoid contamination by agents contained within the vessel. It will thus be appreciated that the present Wharton's jelly extract is essentially free from cord blood cells, umbilical cord epithelial cells, vessel endothelial cells and vessel smooth muscle cells.

Other preferred digestive enzymes and preferred concentrations that can be used in the isolation procedure are, for instance, about 0.1 to about 10 mg/ml hyaluronidase, about 0.05 to about 10 mg/ml trypsin as well as EDTA. The preferred collagenase concentration is about 4 mg/ml for a digestion period of about 3 hours, although a less expensive preferred alternative is to use about 0.5 mg/ml for about 18-24 hours. Still other preferred alternatives to collagenase concentrations are illustrated in FIG. 21. Preferrably, digestion is halted at or before the vessels begins to degrade which, as shown in FIG. 21, occurs at different time points depending on the collagenase concentration.

After about 24 hours in the preferred embodiment of about 0.5 mg/mL collagenase extraction medium, preferably 12-36 hours, and more preferably 18-24 hours, or after the preferred embodiment of about 3 hours in the about 4.0 mg/mL collagenase extraction medium, the vessels are removed, leaving a perivascular tissue extract that contains human progenitor cells. These cells are expanded under conditions standard for expansion of progenitor cells. The cells can, for instance, be selected on polystyrene to select for adherent cells, such as in polystyrene dishes or flasks and then maintained in a suitable culturing medium. In an embodiment of the invention, the extracted cells are cultured for expansion, with or without prior selection for adherent cells, under conditions of stirred suspension, as described for instance by Baksh et al in WO02/086104, the disclosure of which is incorporated herein by reference.

In a particular embodiment of the present invention, the extracted population of HUCPV cells is cultured under adherent conditions, and non-adherent cells resident in the supernatant are recovered for further culturing. These “post-adherent” cells are characterized as a subpopulation by a propensity to form bone nodules and fat cells spontaneously, and constitute a valuable embodiment of the present invention. Thus, in this respect, the present invention further provides an isolated population of progenitor cells extracted from perivascular tissue, the cells having the propensity to form at least one of several differentiated cell types including bone cells, cartilage cells, fat cells and muscle cells, wherein such progenitor cells constitute the non-adherent fraction of the HUCPV cells cultured under adherent conditions. Such cells are obtained by, for instance, the preferred method of culturing the perivascular tissue-extracted HUCPV cells under adherent conditions, selecting the non-adherent cell population, and then culturing the non-adherent cell population under conditions useful to (1) expand said population or (2) to cause differentiation thereof into a desired cell phenotype. Culturing conditions useful therein are those already established for such expansion and differentiation, as exemplified herein.

It will also be appreciated that the present invention includes HUCPV subpopulations that are cultured and expanded under standard adherent culturing conditions. They are thereafter co-cultured with hematopoetic stem cells.

The cells present in the extract can, either directly or after their expansion, be sorted using established techniques to provide expandable subpopulations enriched for cells of a given phenotype. Thus, the present invention further provides perivascular tissue extracted cell populations that are enriched for multipotent mesenchymal progenitor cells, osteoprogenitor cells, cell populations that are enriched for progenitor cells, and cell populations that are enriched for multipotent and osteoprogenitor cells. Preferably, the cells can further be enriched to select for only those that are positive for the pericyte marker 3G5, using antibody thereto, and to select only for those that are negative for either one or both of the major histocompatable complex (“MHC”) class I and class II markers.

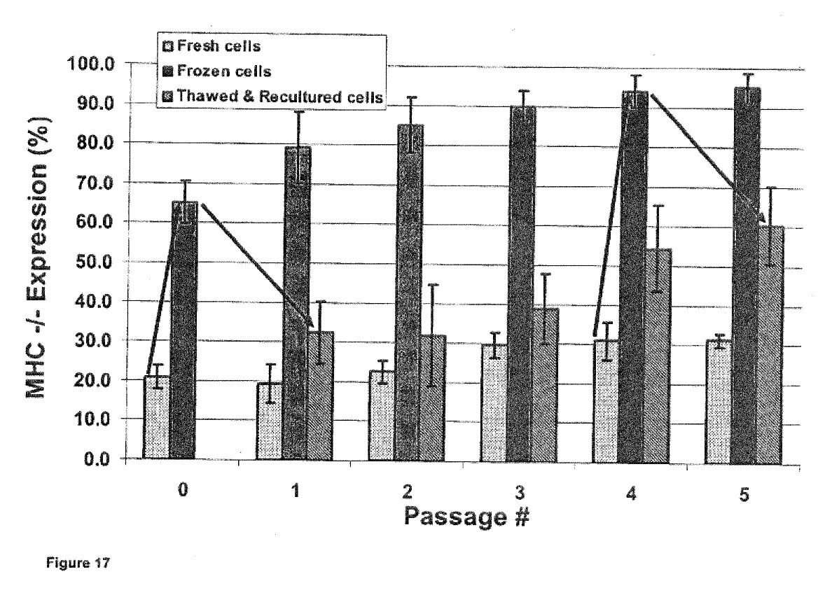

As is revealed in FIG. 17, it has been found that the distribution of MHC markers within the progenitor cell population is altered by freeze-thawing. Upon passaging of fresh cells, the frequency of MHC double negative cells is relatively constant/marginally increased. However, it has been found, as noted in the examples herein, that the frequency of MHC double negative cells in the progenitor population is increased significantly in cells plated following freezing. Thus, in the present progenitor cell population, cells of the MHC double negative phenotype are further characterized by the propensity to increase in frequency following freezing. Such freezing is performed in the usual manner known in the art, for instance by first preparing a cell aliquot, and then storing the cell preparation for the desired period. It will be appreciated that such cells can be stored for many years if desired.

In an embodiment, the present invention thus further provides a method for producing MHC double negative progenitor cells, by obtaining a perivascular tissue extract as herein described, or an MHC double negative-enriched fraction thereof, subjecting the extract or fraction thereof to freezing, and then co-culturing the frozen cells. The resulting cells as noted are potentially useful to induce tissue formation or repair in human subjects.

The cell populations obtained from the co-cultured extract or from a suitably enriched co-cultured fraction thereof, are useful either directly or following their expansion to provide differentiated cell populations. All of the procedures suitable for their fractionation and enrichment, and for their expansion are well established in the art, and are exemplified herein. Expansion can proceed, for instance, in the presence of factors such as IL-3 and Stem Cell Factor, and similar agents known in the art. In one embodiment, the present cell population, and particularly the osteoprogenitor cells therein, are subjected to differentiation using conditions established for the growth of bone tissue therefrom. In a preferred embodiment, a subpopulation of osteoprogenitor cells that arise from the co-culturing of the present progenitor cell population, referred to as committed osteoprogenitors, have the ability to differentiate in the absence of osteogenic supplements. Alternatively, in another preferred embodiment, the osteoprogenitor cells are cultured in a medium supplemented with one or more agents that stimulate osteogenesis, such as dexamethasone. In addition, in yet another preferred embodiment, the co-cultured cells can also be cultured with supplements suitable for stimulating differentiation into other mesenchymally-derived connective tissues (Caplan, 1991), including cartilage, muscle, tendon, adipose etc., all in accordance with standard practice in the art.

As a practical alternative to in vitro culturing of cells in the present cell population, it will be appreciated that in another preferred embodiment, the cells can be transplanted in vivo to induce the formation of a desired tissue directly within a patient.

For use in transplantation, the present cells can be provided as a composition, further comprising a carrier useful for their delivery to the tissue site selected for engineering. The cells are presented in a dose effective for the intended effect. It is expected that a preferred effective cell dose will lie in the range from about 103 to about 10⁷ cells, more preferrably 10⁴-10⁶ cells, and most preferably 2×10⁵ cells, per dose. The carrier selected for delivery of those cells can vary in composition, in accordance with therapeutically acceptable and pharmaceutically acceptable procedures established for delivery of viable cells. In the embodiments, the cells are exploited for purposes of bone tissue engineering. In one embodiment, the cells are presented with a carrier in the form of a scaffold material that serves to localize the cells as an implant at a bone site that is defective or fractured, or is surgically prepared to receive the implant. A variety of materials are suitable as carriers for this purpose. In a particular embodiment, the carrier is formed of resorbable material such as calcium phosphate, PLGA or mixtures thereof. Equivalent materials can be used, provided they allow for the cells to remain viable during formation and delivery of the composition, and are otherwise physiologically compatible at the implantation site.

Still other preferred carriers suitable for delivery of the progenitor cells will include vehicles such as PBS and gels including hyaluronic acid, gelatin and the like with equivalents being useful provided they possess the pH and other properties required for cell viability.

It will also be appreciated that the present cells are useful as hosts for delivering gene expression products to the desired tissue site. That is, the present co-cultured cells can in accordance with embodiments of the present invention, be engineered genetically to receive and express genes that upon expression yield products useful in the tissue repair process, such as the various growth factors which, in the preferred embodiment of bone tissue, can usefully include PTH, the BMPs, calcitonin, and the like. The cells can also be developed as transgenics for other purposes, such as by introduction of genes that alter the cell phenotype, to make it more robust, or more suitable to a given end-use.

Embodiments of the invention are described in the following examples. The examples herein are for purposes of describing embodiments of the invention and are not intended to limit the invention more restrictive than that claimed.

### EXAMPLES

Harvest of Progenitor Cells from Human Wharton's Jelly

The umbilical cord is collected from full-term caesarian section infants immediately upon delivery. The umbilical cord is then transferred by the surgeon into a sterile vessel containing medium (80% .alpha.-MEM, 20% antibiotics).

All procedures from this point on are performed aseptically in a biological safety cabinet. The umbilical cord is washed in Phosphate Buffered Saline (PBS) (—Mg2,—Ca²⁺) three times to remove as much of the umbilical cord blood as possible, and transferred back into a container with medium. A length of approximately 6 cm of cord is cut with sterile scissors and placed onto a sterile cork dissection board. The remaining cord (30-45 cm) is returned to the medium-filled container and placed into an incubator at 37° C. The 6 cm section of cord is ‘twisted’ against its helix, and pinned at both ends to reveal a smooth and straight surface of the umbilical cord epithelium. Using fine scissors, the umbilical cord is cut approximately 1-2 mm deep along its length to reveal the WJ. Starting with each ‘flap’ of cut epithelium, the WJ is teased from its inner surface using the blunt edge of a scalpel, and the teased away epithelium (approximately 0.5 mm thick) is pinned down. This procedure results in the WJ being exposed, and with its three vessels embedded in it running straight from end to end rather than helically along its longitudinal axis. Care is taken to constantly bathe the section with 37° C. PBS. Isolating one of the ends of a vessel with forceps, it is teased away from the WJ along its length until it is free of the bulk of the WJ matrix. Alternatively, the middle of the vessel can be dissected from the matrix, held with tweezers, and teased from the matrix in each direction toward its ends. Once freed by either method, the vessel is surrounded with approximately 1-2 mm of the cell-bearing WJ matrix. The dissected vessel is then clipped at both ends with either a surgical clamp, mosquito clip or sutured to create a ‘loop,’ blocking the passage of fluid either into or out of the vessel. The ‘loop’ is immediately placed along with the scissors into a 50 ml tube containing a 0.5 mg/ml collagenase solution with PBS (—Mg²⁺, —Ca²⁺), and placed into an incubator at 37° C. The remaining two vessels are dissected in a similar fashion, looped, and also placed in the collagenase solution in the incubator. Subsequent to the removal of the vessels, strips of WJ, constituting perivascular tissue, can easily be dissected off the epithelium and placed into 50 ml tubes with the collagenase solution. The remaining epithelial layer is then disposed of in a biohazard waste container. The same protocol is used with the remaining 30-45 cm of umbilical cord, producing 15 to 25 tubes with either ‘loops’ or perivascular tissue strips.

### Initiation of Wharton's Jelly Progenitor Cell Cultures

After 18-24 hours, the ‘loops’ are removed with the aid of their attached suspension clamp or suture and a pipette, and the remaining suspensions are then diluted 2-5 times with PBS and centrifuged at 1150 rpm for 5 minutes to obtain the cell fraction as a pellet at the bottom of the tubes. After removal of the supernatant, the cells are resuspended in eight times volume of 4% NH₄Cl for 5 minutes at room temperature in order to lyse any contaminating red blood cells. The suspensions are then centrifuged again at 1150 rpm for 5 minutes to isolate the cell fraction as a pellet, and the supernatant is removed. After counting the cells with the use of hemocytometer, they are plated directly onto T-75 cm tissue culture polystyrene dishes, and allowed to incubate at 37° C. for 24-72 hours in order to allow the cells to attach to the polystyrene surface. The medium is then changed every two days.

The attached cells are passaged using 0.1% trypsin solution after 7 days, at which point they exhibit 80-90% confluency, as observed by light microscopy, and there is evidence of ‘mineralized’ aggregate formation, as revealed under phase microscopy and indicated by expected changes in optical properties. Upon passage, cells are plated either in 35 mm tissue culture polystyrene dishes or 6 well plates at 4×10³ cells.cm² in supplemented media (SM) (75% α-MEM or D-MEM, 15% FBS, 10% antibiotics) and treated with 10-⁸M Dex, 5 mM β-GP and 50 .mu.g/ml ascorbic acid to test the osteogenic capacity of these cells. These plates are observed on days 2, 3, 4 and 5 of culture for CFU-O otherwise referred to as ‘bone nodule’ formation.

In order to test the chondrogenic capacity of these cells, 2×10⁵ cells are centrifuged at 1150 rpm for 5 minutes in order to obtain the cells as a pellet. Once the supernatant is removed, the cells are maintained in SM supplemented with 10 ng/ml transforming growth factor-beta (TGF-.beta.) (and optionally with 10⁻⁷M dexamethasone). The supplemented medium is replaced every two days, maintaining the cultures for 3-5 weeks, at which point they are harvested for histology (by fixation with 10% neutral formalin buffer (NFB)), embedded in paraffin, cut into 6 .mu.m section, and stained for the presence of collagen II (antibody staining) and the presence of glycosaminoglycans (alcian blue staining). To assess the adipogenic differentiation capacity of the cells, they are initially cultured in 6-well plates in SM (with D-MEM), which is replaced every 2 days, until they reach 60% confluence. At that point the medium is replaced with the adipogenic induction medium (AIM) (88% D-MEM, 3% FBS, 33 .mu.M Biotin, 17 .mu.M Pantothenate, 5 .mu.M PPAR-gamma, 100 nM Bovine insulin, 1 .mu.M Dexamethasone, 200 .mu.M Isobutyl methylxanthine and 10% antibiotics). The AIM is replaced every 2 days for 10 days at which point the cells are fixed in 10% NFB and stained with Oil Red O which stains the lipid vacuoles of adipocytes red. Finally, in order to assess the myogenic capacity of the cells, they are initially cultured in T-75 cm.sup.2 tissue culture flasks in SM (with D-MEM) until they reach 80-90% confluence, at which point the medium is replaced with myogenic medium (MM) (75% D-MEM, 10% FBS, 10% Horse serum, 50 .mu.M hydrocortisone and 10% antibiotics). The MM is replaced every 2 days. After 3-5 weeks in culture, the cells are removed from the culture surface (see subculture protocol), lysed in order to obtain their MRNA, and assessed by rtPCR for the presence of several myogenic genes, including: MyoG, MyoD1, Myf5, Myosin heavy chain, myogenin and desmin.

### Progenitor Assays

### Cell Proliferation Assay

The following cell proliferation assay may be expected from the first cell culture group: During the weekly passage procedure (occurring every 6 days), aliquots of 3×10⁴ cells are plated into each well of 24 6-well tissue culture polystyrene plates. On days 1, 2, 3, 4, 5 and 6 days of culture, four of the 6-well plates are passaged and the cells are counted. The exponential expansion of these cells is plotted, and the mean doubling time for the cells in these cultures is calculated. Results are shown in FIG. 16. It will be noted that the doubling time for the PVWJ cell culture is about 24 hours across the entire culturing period. During the log phase, the doubling time is a remarkable 16 hours. This compares with literature reported doubling times of about 33-36 hours for bone marrow mesenchymal cells (Conget and Minguell, 1999), and about 3.2 days for mesenchymal stem cells derived from adipose tissue (Sen et al., 2001). For observation of proliferation with successive passaging, 3×10⁵ cells are plated into 4 T-75 flasks (n=4) and fed with SM which is replaced every 2 days. After 6 days of culture the cells are sub-cultured (see sub-culture protocol above), and counted with the use of a hemocytometer. Aliquot of 3×10⁵ cells are seeded into 4 new T-75 flasks, cultured for 6 days, and the process of counting is repeated. This process is repeated from P0 through P9 for 4 cord samples.

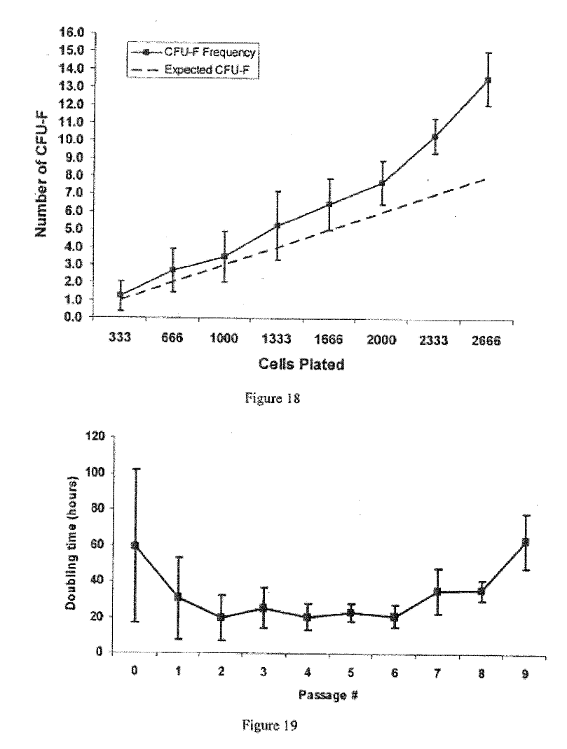

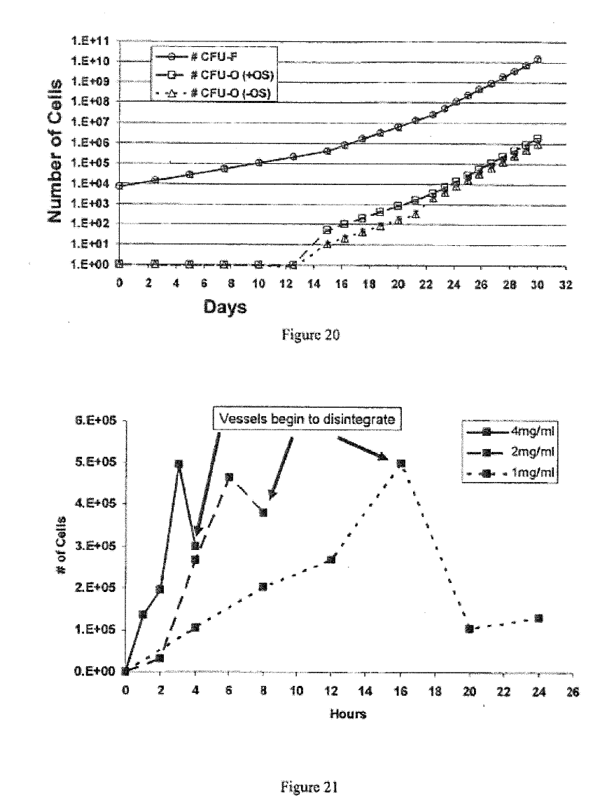

FIG. 18 illustrates the expected CFU-F frequency of HUCPV cells. The frequency of 1:300 is significantly higher than that observed for other mesenchymal progenitor sources including neonatal BM (1:10⁴) (Caplan, 1991), and umbilical cord blood-derived “unrestricted somatic stem cells” (USSCs) (Kogler et al., 2004) which occur at a frequency of 1:2.times.10⁸. FIG. 19 illustrates the proliferation rate of HUCPV cells with successive passaging. The initial doubling time of 60 hours at P0 drops to 38 hours at P1, which drops and maintains itself at 20 hours from P2-P8. The cells begin to enter senescence thereafter and their proliferation rate begins to drop rapidly. Interestingly, when observed during the first 30 days of culture (FIG. 20), HUCPV cells derive 2×10¹⁰ cells within 30 days. As one therapeutic dose (TD) is defined as 2×10⁵ cells (Horwitz et al, 1999) (Horwitz E M, Prockop D J, Fitzpatrick L A, Koo W W, Gordon P L, Neel M et al. Transplantability and therapeutic effects of bone marrow-derived mesenchymal cells in children with osteogenesis imperfecta. Nat Med 1999; 5: 309-313.), HUCPV cells can derive 1 TD within 10 days of culture, and 1,000 TDs within 24 days of culture.

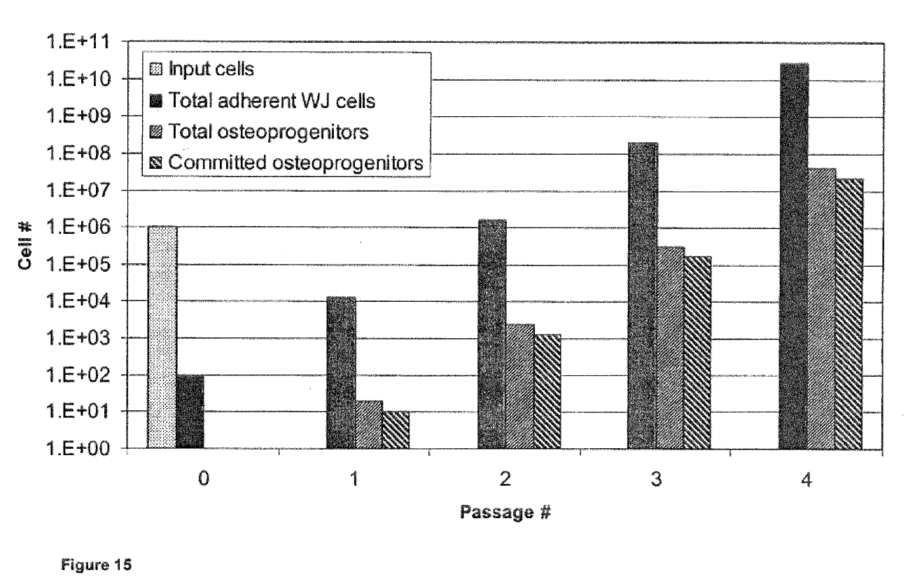

As shown in FIG. 15, the perivascular tissue-derived progenitors comprise different sub-populations of progenitor cells.

Chondrogenic, adipogenic and myogenic differentiation of the cells can be observed.

### Serial Dilution and CFU-F Assays

Dilutions of 1×10⁵, 5×10⁴, 2.5×10⁴, 1×10⁴, 5×10³, 1×10³, HUCPV cells are seeded onto 6-well tissue culture plates (Falcon#353046) and fed every two days with SM. The number of colonies, comprising>16 cells, are counted in each well on day 10 of culture, and confirmed on day 14. CFU-F frequency, the average number of cells required to produce one colony, is consequently determined to be 1 CFU-F/300 HUCPV cells plated. Based on this frequency, the unit volume required to provide 300 HUCPV cells (done in triplicate from each of 3 cords) is calculated, and 8 incremental unit volumes of HUCPV cells are seeded into individual wells on 6-well plates. Again, colonies comprising>16 cells (CFU-Fs) are counted on day 10 of culture to assay CFU-F frequency with incremental seeding.

### Data Analysis

Tetracycline Stain: Tetracycline (9 .mu.g/ml) is added to the cultures prior to termination. At termination, the cells are fixed in Karnovsky's fixative overnight and then viewed by UV-excited fluorescence imaging for tetracycline labeling of the mineral component of the nodular areas.
Scanning Electron Microscopy (SEM): Representative samples of CFU-O cultures are prepared for SEM by first placing them in 70%, 80%, 90% and 95% ethanol for 1 hour, followed by immersion in 100% ethanol for 3 hours. They are then critical point dried. A layer of gold approximately 3 nm layer is sputter coated with a Polaron SC515 SEM Coating System onto the specimens, which are then examined at various magnifications in a Hitachi S-2000 scanning electron microscope at an accelerating voltage of 15 kV. The images generated are used to demonstrate the presence of morphologically identifiable bone matrix.
Flow Cytometry for HLA-Typing of the HUCPV cell population: Test cell populations of >1×10⁵ cells are washed in PBS containing 2% FBS (StemCell Batch #: S13E40) and re-suspended in PBS+2% FBS with saturating concentrations (1:100 dilution) of the following conjugated mouse IgG1 HLA-A,B,C-PE and HLA-DR,DP,DQ-FITC for 30 minutes at 4° C. The cell suspension is washed twice with PBS+2% FBS, stained with 1 .mu.g/mil 7-AAD (BD Biosciences) and re-suspended in PBS+2% FBS for analysis on a flow cytometer (XL, Beckman-Coulter, Miami, Fla.) using the ExpoADCXL4 software (Beckman-Coulter). Positive staining is defined as the emission of a fluorescence signal that exceeded levels obtained by >99% of cells from the control population stained with matched isotype antibodies (FITC- and PE-conjugated mouse IgG1,.kappa. monoclonal isotype standards, BD Biosciences). For each sample, at least 10,000 list mode events are collected. All plots are generated in EXPO 32 ADC Analysis software.

In addition to HLA typing, the HUCPV cell population is also assessed for other markers, with the following results:

1 Marker Expression CD105 (SH2)++CD73 (SH3)++CD90 (Thy1)++CD44++CD117 (c-kit) 15%+MHC I 75%+MHC II−CD106 (VCAM1)−STRO1−CD123 (IL-3)−SSEA-4−Oct-4−HLA-G−CD34−CD235a (Glycophorin A)−CD45−

### Results

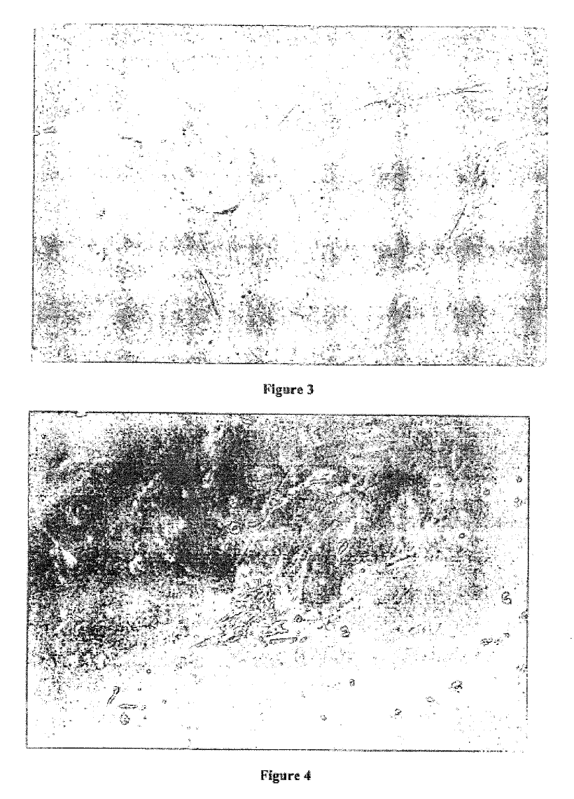

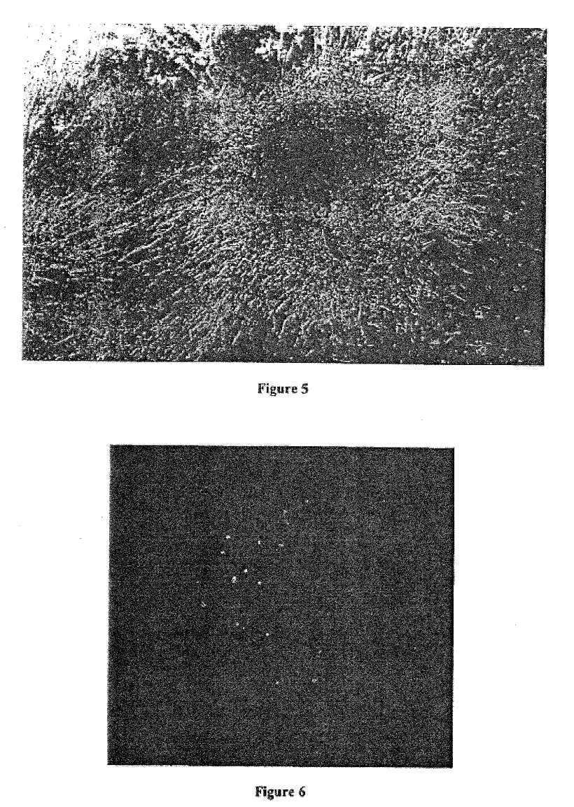

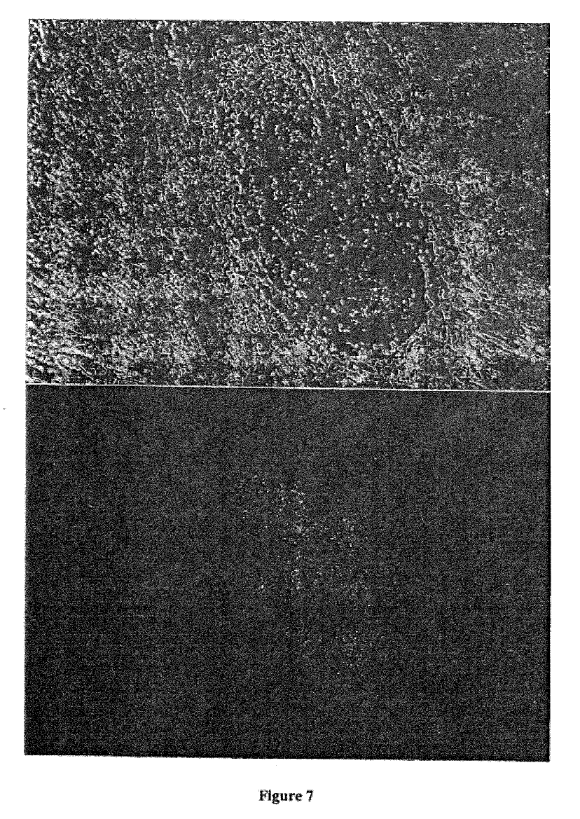

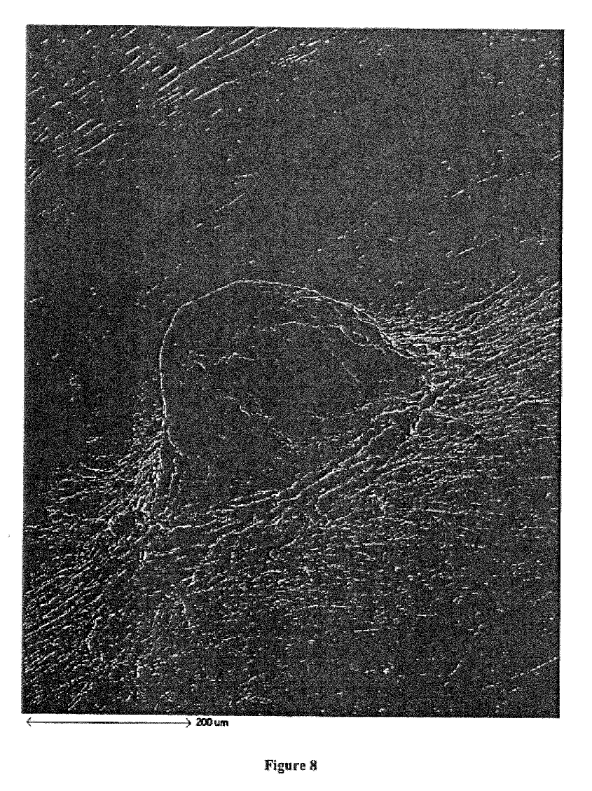

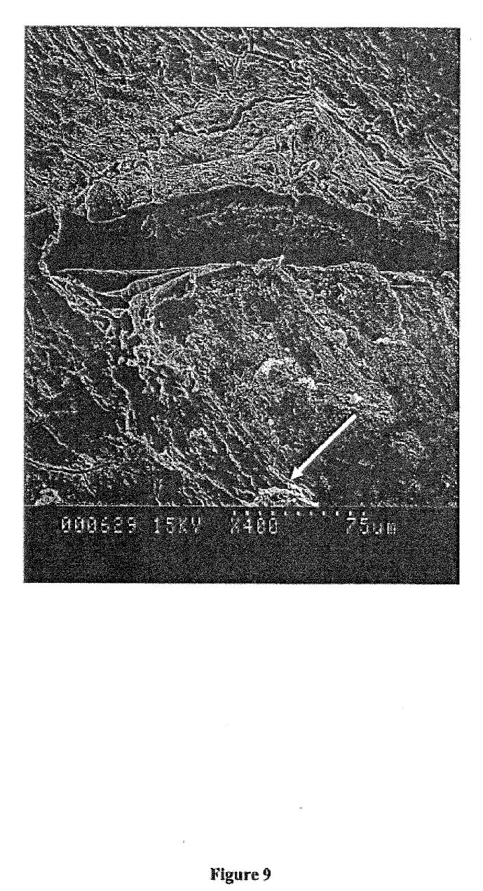

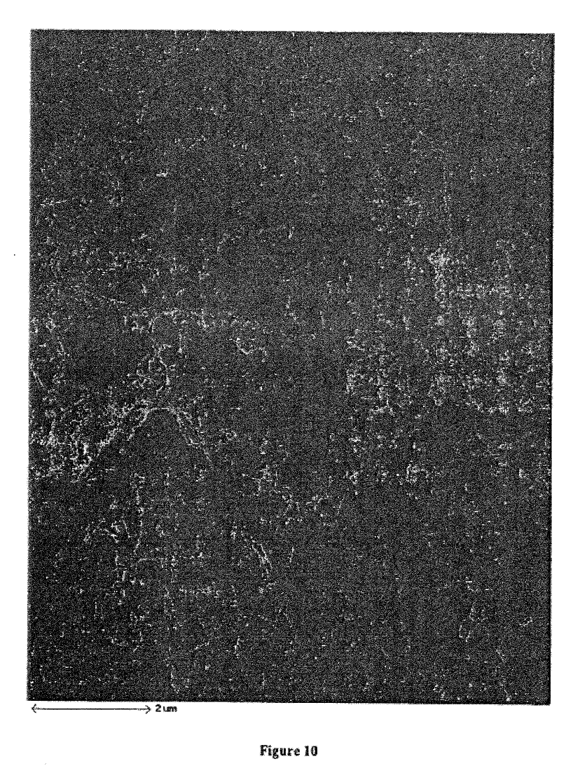

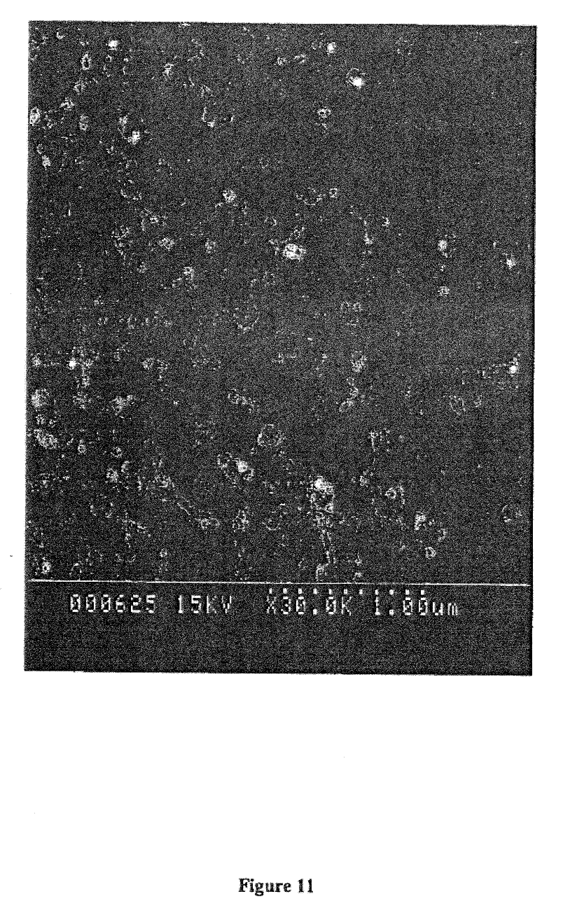

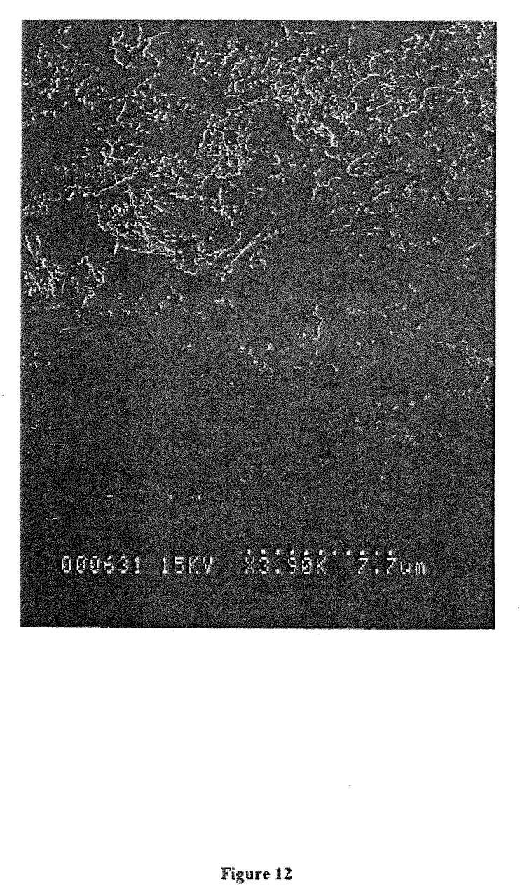

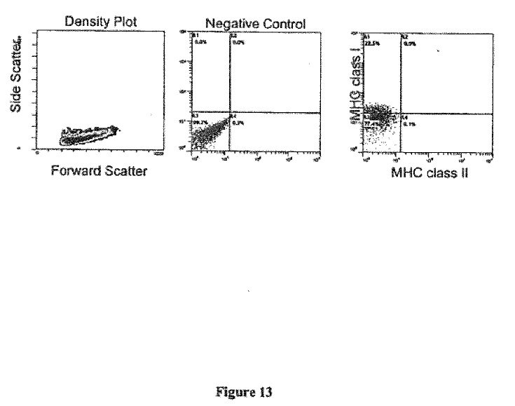

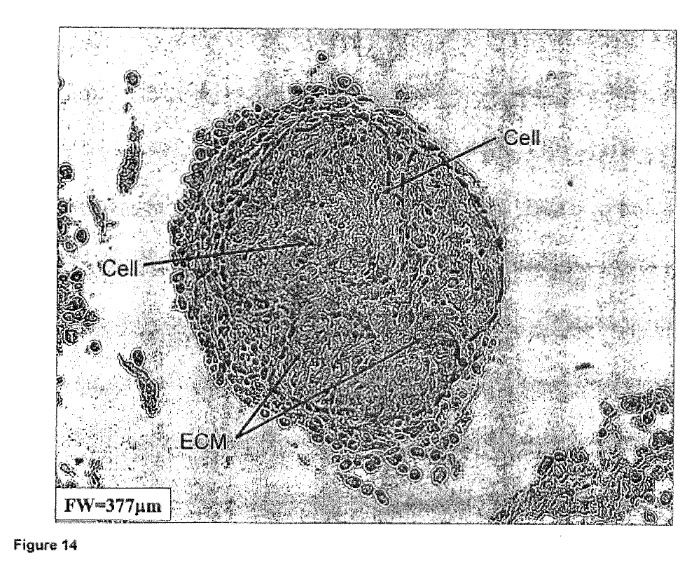

Light Micrographs of Bone Nodule Colonies: FIGS. 3, 4 and 5 illustrate CFU-O's that are present in the cultures on day 3 and day 5. They demonstrated the confluent layer of “fibroblast-like” cells surrounding a nodular area represented by an ‘aggregation’ of polygonal cells that are producing the bone-matrix. These CFU-O's are observed in both the Dex (+) and Dex (−) cultures, and displayed similar morphology over successive passages.
Tetracycline Labeling of CFU-O Cultures: Tetracycline labeling of cultures is used for labeling newly formed calcium phosphate associated with the biological mineral phase of bone. The tetracycline labeling of the cultures coincide with the mineralized nodular areas, which is visualized by exposing the cultures to UV light. FIGS. 6 and 7 depict tetracycline labeled CFU-O cultures of Day 3 and Day 5 cultures of progenitor cells. These images are generated by UV-excited fluorescence imaging, and photographed.
Scanning Electron Microscopy: The CFU-O's are observed under SEM for formation of mineralized collagen matrix which demonstrates the formation of the CFU-O's from the initial stages of collagen formation through to the densely mineralized matrix in the mature CFU-O. FIGS. 8, 9, 10, 11, 12 and 14 represent scanning electron micrographs of the CFU-Os.
Flow Cytometry & HLA-typing of the HUCPV cell population: The flow cytometry, identifying cell-surface antigens representing both Major Histocompatibility Complexes (MHCs) demonstrates 77.4% of the population of isolated cells as MHC^{-/-}. FIG. 13 illustrates the flow cytometry results in relation to the negative control. FIG. 17 shows the impact of freeze-thawing on the frequency of MHC^{-/-} cells in the progenitor population.

### The Effect of Freeze-Thawing

Test cell populations of >1×10⁵ cells is washed in PBS containing 2% FBS and re-suspended in PBS+2% FBS with saturating concentrations (1:100 dilution) of the following conjugated mouse IgG1 HLA-A,B,C-PE (BD Biosciences #555553, Lot M076246) (MHC I), HLA-DR,DP,DQ-FITC (BD Biosciences #555558, Lot M074842) (MHC II) and CD45-Cy-Cychrome (BD Biosciences #555484, Lot 0000035746) for 30 minutes at 4° C. The cell suspension is washed twice with PBS+2% FBS and re-suspended in PBS+2% FBS for analysis on a flow cytometer (XL, Beckman-Coulter, Miami, Fla.) using the ExpoADCXL4 software (Beckman-Coulter). Positive staining is defined as the emission of a fluorescence signal that exceeded levels obtained by >99% of cells from the control population stained with matched isotype antibodies (FITC-, PE-, and Cy-cychrome-conjugated mouse IgG1,.kappa. monoclonal isotype standards, BD Biosciences), which is confirmed by positive fluorescence of human BM samples. For each sample, at least 10,000 list mode events were collected. All plots are generated in EXPO 32 ADC Analysis software.
Sub-Culture & Cell Seeding: The attached cells are sub-cultured (passaged) using 0.1% trypsin solution after 7 days, at which point they exhibit 80-90% confluency as observed by light microscopy. Upon passage, the cells are observed by flow cytometry for expression of MHC-A,B,C, MHC-DR,DP,DQ, and CD45. They are then plated in T-75 tissue culture polystyrene flasks at 4.times.10.sup.3 cells/cm.sup.2 in SM, and treated with 10⁻⁸M Dex, 5 mM βGP and 50 .mu.g/ml ascorbic acid to test the osteogenic capacity of these cells. These flasks are observed on days 2, 3, 4, 5 and 6 of culture for CFU-O or bone nodule, formation. Any residual cells from the passaging procedure also are cryopreserved for future use.
Cryopreservation of Cells: Aliquots of 1×10⁶ PVT cells are prepared in 1 ml total volume consisting of 90% FBS, 10% dimethyl sulphoxide (DMSO) (Sigma D-2650, Lot# 11K2320), and pipetted into 1 ml polypropylene cryo-vials. The vials are placed into a −70° C. freezer overnight, and transferred the following day to a −150° C. freezer for long-term storage. After one week of cryo-preservation, the PVT cells are thawed and observed by flow cytometry for expression of MHC-A,B,C, MHC-DR,DP,DQ, and CD45. A second protocol is used in which the PVT cells are thawed after one week of cryopreservation, recultured for one week, sub-cultured then reanalyzed by flow cytometry for expression of MHC-A,B,C, MHC-DR,DP,DQ, and CD45.
Results: The results are presented in FIG. 17. It will be noted that the frequency of MHC^{-/-} within the fresh cell population is maintained through several passages. When fresh cells are frozen after passaging, at −150° C. for one week and then immediately analyzed for MHC phenotype, this analyzed population displays a remarkably enhanced frequency of cells of the MHC^{-/-} phenotype. Thus, and according to an embodiment of the present invention, cells of the MHC^{-/-} phenotype can usefully be enriched from a population of PVT cells by freezing. Still further enrichment is realized upon passaging the cultures of the previously frozen cells. In particular, and as seen in FIG. 17, first passage of cryopreserved cells increases the relative population of MHC^{-/-} cells to greater than 50% and subsequent freezing and passaging of those cells yields an MHC^{-/-} population of greater than 80%, 85%, 90% and 95%.

### Harvest of Post Adherent HUCPV Cell Fraction

The yield of progenitors recovered from the perivascular tissue can be enhanced in the following manner. In order to harvest the “post adherent” (PA) fraction of HUCPV cells, the supernatant of the initially seeded HUCPV harvest is replated onto a new T-75 flask, and incubated at 37.degree. C., 5% CO.sub.2 for 2 days. The initially seeded HUCPV flask is then fed with fresh SM. After 2 days this supernatant is again transferred to a new T-75 flask, and the attached cells fed with fresh SM. Finally, the supernatant of the third seeded flask is aspirated, and this flask fed with fresh SM. (Consequently, for each cord, 3 flasks are generated: the initially seeded flask, the first PA fraction and the second PA fraction.) Similar to identical characteristics of these cells are seen compared to the initially seeded cells, confirming that higher cell yields are obtained by isolating these PA fractions.

### Expansion in a Bioreactor

Referring now to FIG. 22, the general organization of the present invention is illustrated. A frame means 10 has vertical and spaced apart plates 11, 12 which support a motor pulley 14 and a housing pulley 13 where the pulleys 13, 14 are connected by a belt drive 15. The motor pulley 14 is coupled to a motor 16 which can be controlled in a well known manner to provide a desired drive speed.

The housing pulley 13 is connected to a drive shaft 17 which extends through a rotative coupling 18 to an inlet end cap 20. The inlet end cap 20 is attached to a central assembly 21 and to a tubular outer culture cylinder 22. At the other end of the central assembly 21 and the culture cylinder 22 is an outlet end cap 24.

An air pump 25 on the frame means 10 is connected by input tubing 26 to a filter 27. An output tubing 28 from the pump 25 couples to the rotative coupling 18 where the air input is coupled from a stationary annular collar to an internal passageway in the rotating drive shaft 17.

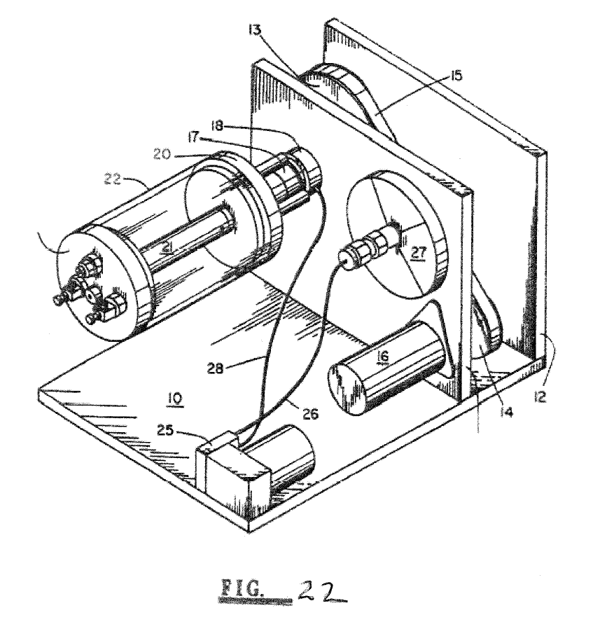

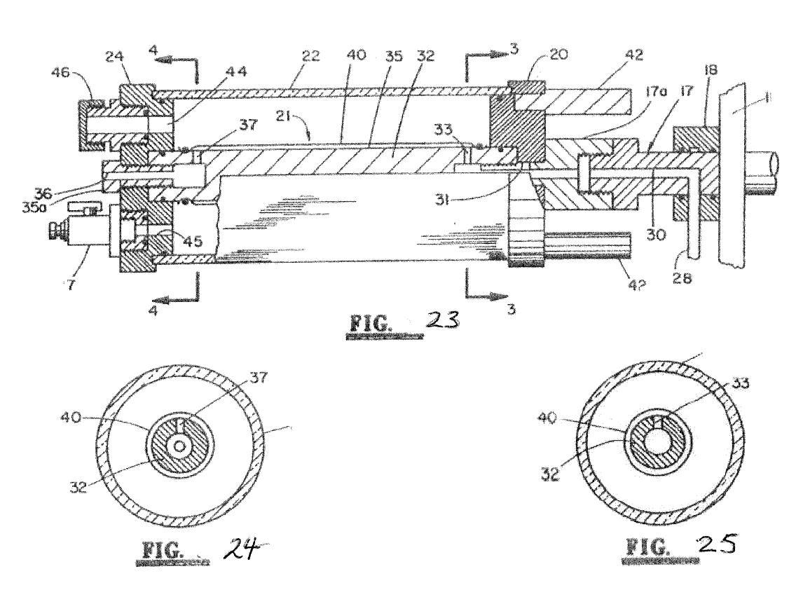

Referring now to FIG. 23, the cell culture system of the present invention is illustrated in partial cross section where the rotative coupling 18 receives the output tubing 28 and the drive shaft 17 has a central air inlet passageway 30 for the passage of air. The drive shaft 17 is attached to a coupling shaft 17a which extends through a central opening 31 in the inlet end cap 20. The coupling shaft 17a is threadedly attached to a cylindrically shaped, central support member 32. The central passageway 30 extends inwardly through the shafts 17, 17a to a transverse opening 33 which couples the air inlet passageway 30 to the exterior surface 35 of the central support member 32. The central support member 32 is sealingly received in a counterbore in the inlet end cap 20 and at its opposite end, the support member 32 is sealingly received in a counterbore of the outlet end cap 24. A tubular outlet member 35a is threadedly attached through a bore in the outlet end cap 24 to a blind bore in the support member 32 and an air exit passageway 36 in the outlet coupling is connected by a transverse opening 37 to the exterior surface 35 of the central support member 32. A tubular oxygen permeable membrane 40 is disposed over the central support member 32 and has its ends extending over the openings 33 and 37 in the central support member 32 so that the membrane 40 can be sealingly attached to the central support member 32 by O-rings or the like. Thus an air passageway is provided for an input of air through the passageway 30 and the transverse opening 33, through the annular space between the inner wall of the membrane 40 and the outer wall of the central support member 32 to the exit transverse opening 37 and to the exit passageway 36. The membrane 40 may be made of silicone rubber which operates under air pressure to permit oxygen to permeate through the wall of the membrane into the annulus of fluid medium surrounding the membrane and carbon dioxide to diffuse in the opposite direction.

Coaxially disposed about the central support shaft 32 is a tubular outer cylinder 22 which can be glass. The cylinder 22 is sealing received on the end caps 20, 24 and defines an annular culture chamber between the inner wall of the cylinder 22 and the outer surface of the membrane 40. On the inlet end cap 20 are circumferentially spaced apart cylindrical members 42. When the coupling shaft 17a is detached from the shaft 17, the members 42 provide a base for standing the cylinder 22 upright or in a vertical position for sampling, changing or adding fluids to the system.

In the outlet end cap 24, there are two or more access ports 44, 45, port 44 having closure means 46 and port 45 being closed by valve 47. A hypodermic needle with fluid medium can be inserted through one access port to inject fluid when withdrawing fluid from the other port. In this regard samples or media can be withdrawn without forming an air space, thereby preserving the zero head space.

One embodiment of the present invention thus involves the central cylindrical core which is a source of oxygenation through the cylindrical membrane and the membrane and outer wall of the vessel are rotated about a horizontal axis. This involves a type of clinostat principal, i.e. a principal that fluid rotated about a horizontal or nearly horizontal axis in one direction, 360°, can effectively suspend particles in the fluid independent of the effects of gravity. The rotational speed of the cylinder 22 effectively eliminates the velocity gradient at the boundary layer between the fluid and the cylinder wall. Thus, shear effects caused with a rotating fluid and stationary wall are significantly reduced or eliminated. In use, an essentially quiescent three-dimensional environment is created in the cylinder.

Co-culturing process
A Wharton's Jelly extract cell mixture was prepared as described above. A hematopoetic stem cell mixture was prepared as follows:

Whole blood is collected from the peripheral circulation, from umbilical cord blood or from cellular product of bone marrow aspirates. The red blood cell component is then removed by isolating the nucleated cell fraction by density gradient separation (Buffy Coat), including by isolating the mononuclear cell fraction (MNC) by layering or Ficoll® or Hetastarch® or other methods, such as immune purification and red blood cell lysis. The buffy coat layer and MNC contain stem cells, progenitor cells and differentiated cells, but the red blood cell component has been removed. In one embodiment of the invention, the entire buffy coat, or the mononuclear cell fraction may be utilized without further manipulation. Alternately, the MNCs are further manipulated by immunomagnetic selection to isolate a specific cell type such as CD133+ or CD34+ cells.

A three dimensional co-culture is initiated in the following manner. The culture device, a slow turning lateral vessel (STLV), is prepared by washing with a tissue culture detergent, (micro.x) and followed by extensive rinses and soaking in Milli Q ultra high purity water. The device is sterilized by autoclaving and upon cooling is rinsed for residuals with culture growth media. The vessel is placed in a laminar flow hood and stood upright. Cytodex 3 microcarrier beads (Pharmacia) are hydrated and sterilized before hand and suspended in a 20 mg/ml solution of growth media; each mg containing 4000 micro carriers. The vessel is filled with the growth media so that there is essentially zero headspace, which consist of minimal essential medium alpha (MEM), supplemented with insulin, transferrin, selenium, (5 ug., 10 ug., 5 ug.), epidermal growth factor, sodium pyruvate, 10% fetal calf serum, hepes buffer 2 grams/liter, and penicillin and streptomycin (100 units, 100 mg./ml.).

62.5 ml of a 20 mg/ml solution of microcarriers is added to the vessel to yield a final concentration of 5 mg/ml. of microcarrier in the vessel. The vessel is then filled within 10% of the final volume with growth media. The vessel is sealed and placed in a laminar flow CO₂ incubator with 95% air, 5% CO₂, 95% humidity at 37° C. to equilibrate for one hour. At the end of one hour, the vessel is removed from the incubator and inoculated with approximately 5×10⁷ cells of equal portions of the WJ mixture and hematopoetic cell mixture. The cells were mixed in a (9:1) ratio. After inoculation, the vessel is closed, purged of remaining air bubbles and replaced in the incubator. The vessel is equipped with a 20 ml. syringe which functions as a compliant volume. Daily monitoring of the growth in the vessel is accomplished by analysis of DCO2, DO2, glucose, mOsm and PH. At 48 hours the growth media is replaced for the first time and each 24-hours thereafter a media change is required. These changes are required to remove toxic metabolic by-products and replenish nutrient levels in the vessel. Media changes are also necessary to harvest rare growth products produced from the interaction of the multicellular organoid culture. On day 2 the rotation rate is increased from 12 to 15 RPM. At 168 hours the media composition is altered to include an additional 100 mg./dl. glucose as a result of increased consumption. At 216 hours the glucose concentration is increased to 300 mg/dl again due to the high rate of consumption. From 138 hours on the culture exhibited cell to cell organization. The culture is terminated at 288 hours to begin analysis of the well developed co-culture contained in the vessel. The growth media from the vessel is harvested and placed at −80° C. for future analysis.

The following references are incorporated herein by reference:
Aubin, J E, 1998, Bone stem cells: J Cell Biochem Suppl, v. 30-31, p. 73-82.
Canfield, A E, M J Doherty, B A Ashton, 2000, Osteogenic potential of vascular pericytes, in J E Davies (ed), Bone Engineering: Toronto, EM Squared, Inc., p. 143-151.
Caplan, A I, 1991, Mesenchymal stem cells: J Orthop. Res, v. 9, p. 641-650.
Chacko, A W, S R M Reynolds, 1954, Architecture of deistended and nondistended human umbilical cord tissues, with special reference to the arteries and veins.: Carnegie Institution of Washington, Contributions to Embryology, v. 35, p. 135-150.
Conget, P, J J Minguell, 1999, Phenotypical and functional properties of human bone marrow mesenchymal progenitor cells: J. Cell Physiol, v. 181, p. 67-73.
Haynesworth, S E, D Reuben, A I Caplan, 1998, Cell-based tissue engineering therapies: the influence of whole body physiology.: Adv Drug Deliv Rev, v. 33, p. 3-14.
Kogler, G, S Sensken, J A Airey, T Trapp, M Muschen, N Feldhahn, S Liedtke, R V Sorg, J Fischer, C Rosenbaum, S Greschat, A Knipper, J Bender, O Degistirici, J Gao, A I Caplan, E J Colletti, G Almeida-Porada, H W Muller, E Zanjani, P Wernet, 2004, A new human somatic stem cell from placental cord blood with intrinsic pluripotent differentiation potential: J. Exp. Med., v. 200, p. 123-135.
Mitchell, K L, M L Weiss, B M Mitchell, P Martin, D Davis, L Morales, B Helwig, M Beerenstrauch, K Abou-Easa, T Hildreth, D Troyer, 2003, Matrix cells from Wharton's jelly form neurons and glia: Stem Cells, v. 21, p. 50-60.
Parry, E W, 1970, Some electron microscope observations on the mesenchymal structures of full-term umbilical cord: Journal of Anatomy, v. 107, p. 505-518.
Pereda, J, P M Motta, 2002, New advances in human embryology: morphofunctional relationship between the embryo and the yolk sac: Medical Electron Microscopy, v. 32, p. 67-78.
Romanov, Y A, V A Svintsitskaya, V N Smimov, 2003, Searching for alternative sources of postnatal human mesenchymal stem cells: Candidate MSC-like cells from umbilical cord: Stem Cells, v. 21, p. 105-110.
Schoenberg, M D, A Hinman, R D Moore, 1960, Studies on connective tissue V, Feber formation in Wharton's Jelly.: Laboratory Investigation, v. 9, p. 350-355.
Sen, A, Y R Lea-Currie, D Sujkowska, D M Franklin, W O Wilkison, Y D Halvorsen, J M Gimble, 2001, Adipogenic potential of human adipose derived stromal cells from multiple donors is heterogeneous: J. Cell Biochem., v. 81, p. 312-319.
Takechi, K, Y Kuwabara, M Mizuno, 1993, Ultrastructural and immunohistochemical studies of Wharton's jelly umbilical cord cells: Placenta, v. 14, p. 235-245.
Tuchmann-Duplessis, H, G David, P Haegel, 1972, Illustrated Human Embryology, New York, Springer-Verlag, p. 54-61.
Weiss, L, 1983, Histology: cell and tissue biology, New York, Elseiver Biomedical, p. 997-998.
Wharton, T W, 1656, Adenographia, Translated by Freer S. (1996). Oxford, U.K., Oxford University Press, p. 242-248.

We claim:

1. A process of producing a Wharton's jelly extract composition comprising the steps of:
providing a human umbilical cord with vasculature;
isolating the perivascular tissue proximal to the vasculature;
digesting the perivascular tissue so that fractions are created; and
co-culturing at least a fraction of the digested tissue with hematopoeitic stem cells to produce a Wharton's jelly extract composition comprising progenitor cells.

2. The Wharton's jelly extract composition produced by the process of claim 1.

3. A Wharton's jelly extract composition, wherein the extract comprises human progenitor cells and is obtained by enzymatic digestion of the perivascular tissue proximal to the vasculature of human umbilical cord and thereafter co-cultured with hematopoetic stem cells.

4. A Wharton's jelly extract according to claim 3, wherein the extract is obtained by subjecting umbilical cord vasculature bearing proximal Wharton's jelly to enzymatic digestion in a suitable cell extraction medium.

5. A method for making a composition of tissue regenerating cells comprising obtaining a human progenitor cell, isolating said cell from the Wharton's extract according to claim 1 or 3 and co-culturing said cell with hematopoetic stem cells.

6. A method as in claim 1 or 3 wherein the co-culturing is accomplished in a two-dimensional system.

7. A method as in claim 1 or 3 wherein the co-culturing is accomplished in a rotating vessel bioreactor.

8. A composition of matter comprising an expanded mixture of Wharton's jelly extract composition and hematopoetic stem cells.

9. A method for growing cell cultures including the steps of:
providing a bioreactor comprising: an elongated tubular culture vessel; end caps enclosing the ends of said culture vessel; a shaft co-axially disposed in said culture vessel and extending between said end caps; and a tubular membrane disposed over said shaft between said end caps and sealed with respect to said shaft for defining an annular passageway between said membrane and said shaft and for defining an annular culture chamber between said membrane and the inner wall of said culture vessel, said membrane being oxygen permeable for exchange of component gases with said culture chamber;
completely filling said culture chamber with a fluid nutrient medium containing discrete suspension material and a cell mixture of Wharton's Jelly derived cells and hematopoetic stem cells, said suspension material having a different density from the density of the fluid nutrient medium;
rotating said culture vessel, said shaft and said membrane about the longitudinal axis of said culture vessel, in one direction, said longitudinal axis being horizontally disposed;
controlling the rotation of said culture vessel so as to place the discrete suspension materials and the cell mixture in suspension at spatial locations in the fluid nutrient medium out of interference relationship with one another by virtue of the rotation; and
during said rotation, continuously introducing an oxygen containing gas under pressure at one end of said annular passage and exiting the gas at the other end of said annular passageway.

10. A process of producing a Wharton's jelly extract composition that is prevented from having a significant number of cord blood stem cells therein, comprising the steps of:
providing a human umbilical cord with vasculature;
isolating the perivascular tissue proximal to the vasculature; and
digesting the perivascular tissue so that fractions are created; the improvement comprising
co-culturing at least a fraction of the digested tissue with hematopoietic stem cells to produce a Wharton's jelly extract composition comprising progenitor cells.
